(19) United States
(12) Patent Application Publication
(10) Pub. No.: US 2017/0026747 A1
(43) Pub. Date: Jan. 26, 2017

(54) INTEGRAL ENHANCEMENT OF ORGANIC AND INORGANIC MATTERS USING SPECIFIC TONAL AND VIBRATIONAL LEVELS

(71) Applicant: C. Michael Scroggins, Richmond, TX (US)

(72) Inventor: C. Michael Scroggins, Richmond, TX (US)

(21) Appl. No.: 14/807,078

(22) Filed: Jul. 23, 2015

(51) Int. Cl. H04R 3/04; H03G 5/16; A61M 21/00; H02J 3/38

(52) U.S. Cl. CPC H04R 3/04; H02J 3/383; H03G 5/165; A61M 21/00; A61M 2021/0027

(57) ABSTRACT

Disclosed herein are technologies related to a device to charge a molecular structure of an organic or inorganic matters. This Abstract is submitted with the understanding that it will not be used to interpret or limit the scope or meaning of the claims.

### BACKGROUND

Therapeutic healing practices such as Eastern Healing Arts and Native American Healing Arts have been around for centuries and they work on a principle of subtle energy manipulation. Prayers, Solfeggio tones, Charka tones, etc. utilizes a particular range of tones, for example, that may be traced back to individual areas of a human body. These tones may make up intonation sounds and rate of vibrations for specific organs in a body of a human being, or a portion of organic and inorganic matters according to texts.

On the other hand, magnetic fields produced by Earth and overlapping man-made magnetic fields may further play a part for these tones. That is, a matter that is vibrating at an extremely high level may impact surrounding areas even though the matter has long since outlived vibrational patterns and speed of the surrounding areas. Taking all of this into considerations, the human being, organic and inorganic matters are not evolving at the same rate as the surrounding areas or nature does. In other words, a balance misalignment is created on the organic and inorganic matters and the principle of subtle energy manipulation has thrived up to present generation.

### BRIEF DESCRIPTION OF THE DRAWINGS

FIG. 1 shows an overview of an example operation in accordance with one or more implementations described herein.

FIG. 2 illustrates an example system in accordance with one or more implementations described herein.

FIG. 3 illustrates an example generation unit component in accordance with one or more implementations described herein.

FIG. 4 illustrates an example rod structure of a generation unit component in accordance with one or more implementations described herein.

FIG. 5 illustrates an example process in accordance with one or more implementations described herein.

FIG. 6 illustrates an example computing device to implement in accordance with the technologies described herein.

The Detailed Description references the accompanying figures. In the figures, the left-most digit(s) of a reference number identifies the figure in which the reference number first appears. The same numbers are used throughout the drawings to reference like features and components.

### DETAILED DESCRIPTION

Disclosed herein are technologies related to a device that fills up a gap in a molecular structure or construction of an organic or inorganic materials such as, but not limited to, a human being, a tangible object, plants, animals, picture portraits, oil rigs, bridge structures, base station transmitters, power supply, and other forms of organic or inorganic matters.

For example, a typical inorganic material such as a picture portrait has a molecular structure that includes some gaps due to dead cells on a matrix of paper where the picture portrait is printed upon. With this gap within the molecular structure of the picture portrait, its aging process is accelerated such that the picture portrait will fade in time or decline in quality. As described herein, the device is configured to detect and measure an amount of gap within the molecular structure of the picture portrait. The detecting and measuring of the amount of gap, for example, is implemented in order to overcome or stop the eventual fading or decline in quality of the picture portrait. In this example, the amount of gap includes a subtle change from one state to the next.

For the above picture portrait or music, for example, the picture portrait or music is less impactful if the portrait or sound does not have the correct spacing to be visually or audibly pleasing. That is, slight changes to musical composition or painting may create a less appealing effect due to the gap presence and to this end, filling the amount of the gap may overcome the less appealing effect.

To determine the presence and the amount gap in the picture portrait, the device infuses a sequence of magnetically realigned energy to the picture portrait. The magnetically realigned energy, for example, includes opposing magnetic fields that create minute fields of multiple three dimensional fields that react to the picture portrait, sound, and the like. Based from the infused realigned energy, a transducer may be utilized to compare, for example, the measured resistance along different points or areas in the picture portrait to a pre-configured threshold. With these measurements, the device may be able to detect the areas or points where the gap is located within the molecular structure of the picture portrait.

Furthermore, the device may be able to estimate an amount of charging energy that it may supply to fill the gap. The charging energy, for example, is a field that creates a balance in order to allow smooth transitions between sequences of change in materials, colors or sounds. The charging energy, in this example, may depend upon the amount of magnetically realigned energy that the device is generating per unit time.

In another example, the device may utilize and transform the gap-free picture portrait for purposes of infusing its molecular structure equivalent to another recipient such as a human being. For example, the transformation includes determining an equivalent vibrational rate of the molecular structure of the gap-free picture portrait. The equivalent vibrational rate is a rate at which the central mass is balanced and signal changes occur in an outward radiation from a center point of the central mass until the signal or energy level starts to weaken. From this point where the signal or energy starts to weaken, a new frequency and energy range is formed by the device. In this example, the equivalent vibrational rate may include an energy level that is appropriate for enhancing balance in the molecular structure of the human being.

In another example still, the device may be configured to boost the level of reaction of the human being's molecular structure and at the same time, a musical input to the device is processed and radiated by the device to communicate its calming effect to the human being. In this example, the device will first radiate a magnetically realigned energy towards the human being in order to increase the level of reaction in the molecular structure of the human being. Simultaneous with this radiation, a classical song may be inputted to the device and the device transforms the classical song to its equivalent vibrational rate. Thereafter, the device will communicate the equivalent vibrational rate either through a speaker sound system or through a wired medium that is attached to the human being. The equivalent vibrational rate, for example, may include a frequency that is beyond audible frequency response of the human being i.e., cannot be heard by sense of hearing. In this example, the equivalent vibrational rate generates tranquility and treatment to the recipient human being.

The sounds are far above and/or below the human hearing range. These sounds while not heard do still exist in the natural environment. Submitting these sounds back into the gaps caused by electronic devices allows stress and tension to be relived. It is also possible to align the circadian rhythm in humans and plants by creating a sequenced timeframe (e.g., 4 times a day for humans once every 6 hours and 6 times a day for plants once every 4 hours.) These cycles are typically 30 minutes long. This corresponds with the time cycles for microbial reproduction in the human body and soil as needed to replenish the needed nutrients to accomplish a balance between organic symbiotic relationships.

### Typical Organic and Inorganic Materials

The elemental composition of typical organic or inorganic materials can be looked at from the point of view of atomic composition.

For example, as a general description of the set of materials that are the subject of the implementations described herein, a range or bound in which any material acts or react to is utilized as a point of reference changes. That is, all matter or materials react to frequencies in their complete state. For example, in case of hydrogen, as a complete molecule and as individual entities, the nucleus and electron as a whole are about 0.000127 nanometer and this corresponds to the frequency of the hydrogen as a whole. With this measurement, there is another electron frequency size (about 100^{th} of the total size of the hydrogen structure) and the nucleus about one 10^{th} the size of the whole particle. Furthermore, there is a span of potential frequencies between the nucleus and electron that hold the electron and nucleus apart. This entire sequence is a balance point when these two simple parts of the whole are in balance at the correct speed and they continue to react and interact with their surroundings.

In a case where the hydrogen characteristics combine with more like hydrogen particles, the combination generates an increase in size, slow in speed, and may further share electrons across orbits. With this combination, the resulting overall mass has a larger frequency; however, each individual particle maintains the same defined frequency of its original size. The larger frequency in the resulting overall mass is the stage where particles can shift, combine and become new atomic structures.

As described herein, the device allows the atomic matter of hydrogen to balance within confines of the frequency range 0-1,200 Hz, and thereafter reset the balance points. Balance points refer to the point at which any structure has reached its critical mass. All matter is in a state of composition or decomposition, entropy is never reached and held in stasis. Some implementations fill the minute molecular voids that occur due to the rapid changes the natural environment can make. All organic and inorganic material has to have access to these coded changes as they occur to allow the composition or decomposition to occur in the same format.

### Operation of an Example Device for Energy Enhancement

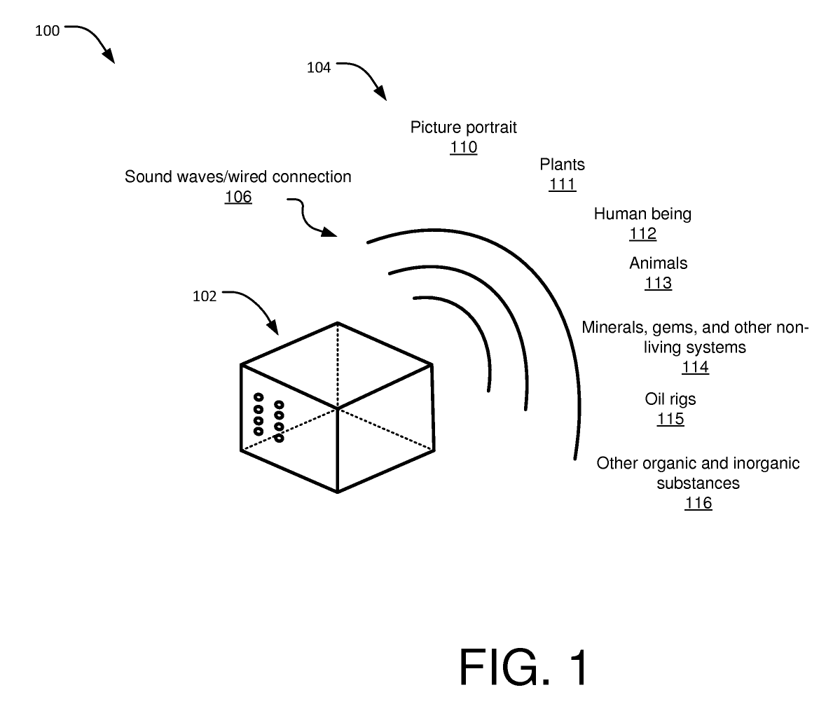

FIG. 1 illustrates an example operation set 100 for implementing, at least in part, the technology described herein. In particular, the operation set 100 depicts a device 102 that is configured to infuse specific frequencies or sequences of random patterned visual and auditory signatures to a recipient 104 by supplying, for example, the magnetically realigned energy through a sound wave or a wired connection 106. The specific frequencies, for example, are within 0-1,800 Hz range while the random patterned visual and auditory signatures are secondary pulses that will send random non-sequential patterns as a subtle reminder to allow a balanced reaction in the recipient 104.

Generally, the recipient 104 may be divided into organic and inorganic materials. For example, the recipient 104 may include, but is not limited to, a picture portrait 110, plants 111, a human being 112, animals 113, minerals/gems/non-living systems 114, oil rigs 115, and other forms of organic/inorganic substances and soil/fertilizer in natural and manmade combinations.115.

For the organic materials, the device 102 is configured to generate sequential frequencies at varied speed (i.e., magnetically realigned energies) to saturate cellular structures of the organic material and the microorganisms/bacteria in soils, streams and oceans a level that allows full absorption without depletion of the nutrients. The saturation of the cellular structures increases a level of reaction of the organic material and with the increased level of reaction, a simultaneous infusion of random patterned music sound wave, for example, may enhance balance in the reaction of the organic material. For example, a person (i.e., human being 112) receives an energy signal in the form of vibrational pulse sequences from the device 102. The received energy signal, in this example, includes random non-sequential pulse that facilitates and/or readies positive changes in a body of the human being the reaction of electronic pules in the human/animal/plant determines the rate of genetic change present in the organism. These resonant values drive the correction of gaps that have occurred in sequential misfiring of synapse 112.

At the same time, a music input to the device 102 facilitates a generation of a non-audible sound wave by the device 102. As described herein, the recipient person or the body of the human being 112 may experience the emotional equivalent of hearing the actual audible music input even though the frequency of the music input was varied to non-audible sound wave frequencies.

For non-organic materials, the device 102 is configured to generate the magnetically realigned energies to fill the gap between molecular combinations within the inorganic materials. The gap, for example, is in a range of more than 0.000127 nanometers but not beyond 0.00000100 nanometers. In this example, the 0.000127-0.00000100 nanometers—range is an effective buffer or change over points to allow separation and consistent metamorphic point of definition between the atomic structures.

By filling the gap in the non-organic materials, the level of reaction of the inorganic materials is boosted so that its sub-atomic particles may be controlled to avoid breakdown, fatigue, increased rate of deterioration, and the like. The sub-atomic particles, for example, follow a pattern of a specific range and size for each part of the cell, and as such, each cell is made up of specific chains of particles from the periodic chart. In this example, infusing frequencies at rates of origin allows the sub-atomic particles to form stronger internal bonds since they are forced into a tighter and more consistent energy charged patterns.

For example, the device 102 is configured to generate a certain level of random frequency sequence to drive the sub-atomic particles in like format (e.g., hydrogen) within the picture portrait 110 until these sub-atomic particles slow down and subsequently combine with larger molecular structures. In this example, the combination fills up the gap between the molecular combinations within the picture portrait 110 and consequently, the picture portrait 110 may be preserved in its present form and state as long as the this molecular combination balance is maintained

Herein, balanced refers to reaching a critical mass potential and can support consistent reproduction of the cellular make up of strong repeatable cells. An imbalance occurs when the critical mass is below the 22.5% mass needed to sustain consistent repeatability. Cancer can reach 22.5% critical mass and quickly morph into the next closest region by slightly changing its makeup, just far enough out of balance that it is not the cell structure it is emulating, but close enough to trick the body into feeding it as a recognizable cellular mass. The maintained molecular combination balance includes the state at which the sub-atomic particle or combination of particles operates the most efficiently. That is, it is the point at which the energy levels are reacting in a smooth flowing pattern from highest to lowest and energy outputs.

### Example System

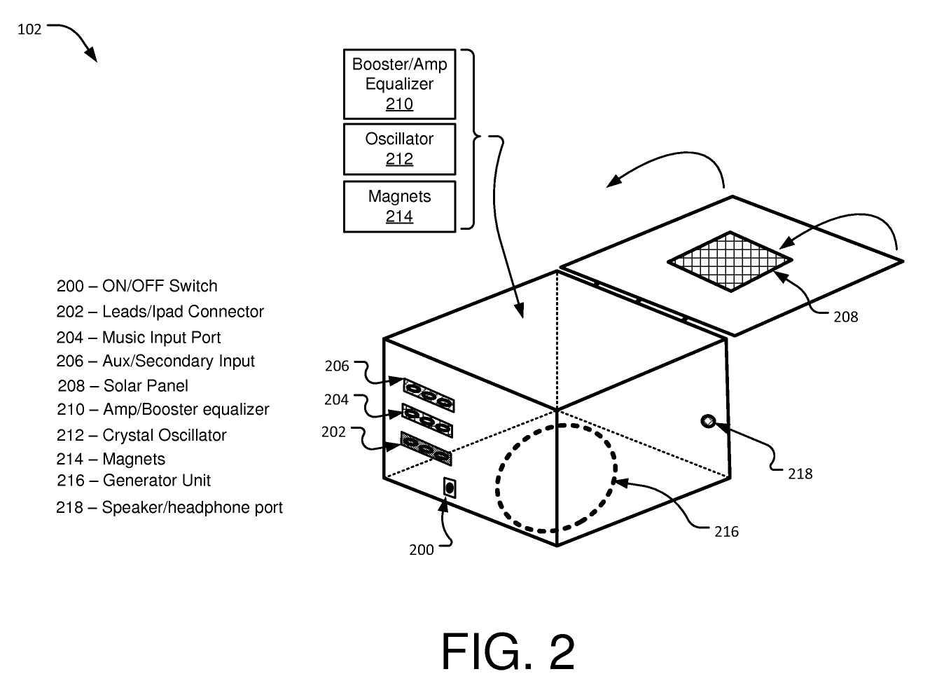

FIG. 2 shows an example system for the device 102 as described in present implementations herein. Generally, the example system of the device 102 includes an input section for receiving an energy, a processing section for processing the received energy, and an output section that outputs the processed energy such as the magnetically realigned energy. As shown, the input section may include a power switch 200, a lead/iPad connector 202, a music input port 204, an auxiliary/secondary input port 206, and a solar panel 208 that is disposed on a lid of the device 102. Furthermore, the processing section may include a booster/amplifier equalizer 210, a crystal oscillator 212, magnets 214, and a generation unit 216; while the output section may include a speaker/headphone port 218.

For the picture portrait example as discussed in FIG. 1 above, the booster/amplifier equalizer 210, crystal oscillator 212, magnets 214 and the generation unit 216 are connected in parallel to generate the magnetically realigned energy that fills the detected gap within the picture portrait (e.g., picture portrait 110). At the output portion, the device 102 transmits, charges, and/or infuses the magnetically realigned energy (i.e., sequences of information) through the speaker/headphone port 218.

For example, a hydrogen atom is a prevalent component in the molecular structure of the picture portrait 110. The hydrogen atom, in this example, is equivalent to 0.0836 Angstroms or 0.00836 nm. Converting 0.00836 nm size to a frequency wavelength, the hydrogen atom is about 2.78×10⁻²⁰ Hertz. In other words, a 60 Hertz operating frequency is about 21.6 times larger than the hydrogen atom and as such, the 60 Hertz frequency would take 22 (πr²)10⁻²⁰ seconds to create one hydrogen that fills a gap area of one nm (10⁻²⁰). For the 0.000127-0.00000100 nanometers—gap range as mentioned above, the 60 Hertz frequency would take a substantially lesser time than the 22 (πr²)10⁻²° seconds to fill the gap range. The lower the frequency the more gaps there are possible, 60 Hz would leave open gaps that need to be filled because more varied molecular structures can travel in a less dense pattern. Some implementations fill the gaps, like of it like a digital sound track, it sounds crisp and clear but it lacks the natural sounds that fill in the gaps. Some implementations of the device have a filler track to music and videos. More of an emotional and physical affect.

Using one single hydrogen atom as a catalyst, the processing section (i.e., booster/amplifier equalizer 210, crystal oscillator 212, magnets 214 and the generation unit 216) of the device 102 may start building blocks or energy signal at a rate of 2.78×10⁻²⁰ Hertz per hydrogen atom. For example, the generation unit 216 can be configured to generate a calculated amount of magnetic fields and at a pre-configured frequency in order to create another hydrogen atom that fills the gap within the molecular structure of the picture portrait 110. In this example, the calculated amounts of magnetic fields at the pre-configured frequency (i.e., magnetically realigned energies) strengthens the portion of the paper where the dead cells are located (i.e., gap).

A pre-configured frequency is taking the molecular structure to its smallest amount, proton, neutron, electron or nucleus. Each of these has a specific size and that size equates to a frequency size (e.g., vibrational energy rate). The sum of the whole atom (e.g., atomic structure) was not as important as the preconfigured frequency size of the sum total of any single part of the atom. As for the excitation rates of hydrogen in water it is not just the 60 Hz and 90 Hz. It is the rate of impact frequency waves have as they cross each other at various points.

The pre-configured frequency, for example, is a frequency that would create a rate of excitation in order to safely release excess unbalanced amount of hydrogen from the gap in the recipient 104. For example, pre-configured frequency rates of 60 Hz and 90 Hz at a power amplitude of 0.05 Volts may create the rate of excitation that pushes the hydrogen from the water. In this example, the excess hydrogen may bubble across the top of the water until it is either absorbed by the atmosphere and the water is considered to be balanced, or the frequency rates are turned off and a subsequent recombination may create the gap-imbalance state again. Similarly, this example applies to humans, plants and animals that hold imbalanced food and water in their respective fat cells. In this case, the application of the pre-configured frequency creates a separation point that safely releases the useless additives from the cellular makeup.

At the input section, the leads/iPad connector 202, music input port 204, auxiliary/secondary input port 206, and the solar panel 208 can be configured to receive energy that is processed to output the magnetically realigned energy at the output port. For example, the solar panel 208 receives an energy (e.g., vitamin D) from exposure to sunlight. This energy is amplified by the booster/amplifier equalizer 210 and the amplified energy is subsequently processed through the crystal oscillator 212 to provide a prism effect (i.e., varying frequency oscillation). In this example, the amplified energy with varying frequency oscillation is then exposed to the magnetic fields that may be generated by the generation unit 216 in combination with the magnets 214.

At this point, the exposure of the amplified energy with varying frequency oscillation to the magnetic fields produces magnetically realigned energies that are capable of filling the gap in the molecular structure of the picture portrait 110. The magnetically realigned energies may be outputted through the speaker sound system, or through a wired connection that may be attached to the picture portrait 110. To make sure that the picture portrait 110 has a gap-free molecular structure, the auxiliary/secondary input port 206 may receive a signal from a transducer (not shown) that is configured, for example, to measure different resistances, frequencies, amplitude, phases, etc. within different areas or points in the picture portrait 110. In this example, the auxiliary/secondary input port 206 may act as a feedback loop for the device 102.

In the case of a human being 112, the processing portion may similarly generate a sequence of combination to boost a level of reaction of the molecular structure in the tissues, muscles, and other parts of the human being 112. Simultaneous with this generation of sequence of combination, the speaker/headphone port 218 may further emit a magnetically realigned energy that corresponds to the molecular structure of the inputted song at the music input port 204 or the auxiliary/secondary input port 206.

For example, the music input port 204 receives a classical song that is supposed to have a calming effect on a listener (e.g., human being 112). In this example, the digital amplitude and frequency components of the classical song can be transformed by the processing portion to its equivalent vibrational rates or equivalent magnetically realigned energies. The magnetically realigned energies may include non-audible frequencies (i.e., beyond human being's hearing range) and are calculated to deliver the calming effect of the classical song even though the recipient human being 112 may not be able to hear or feel it.

### Example Generation Unit

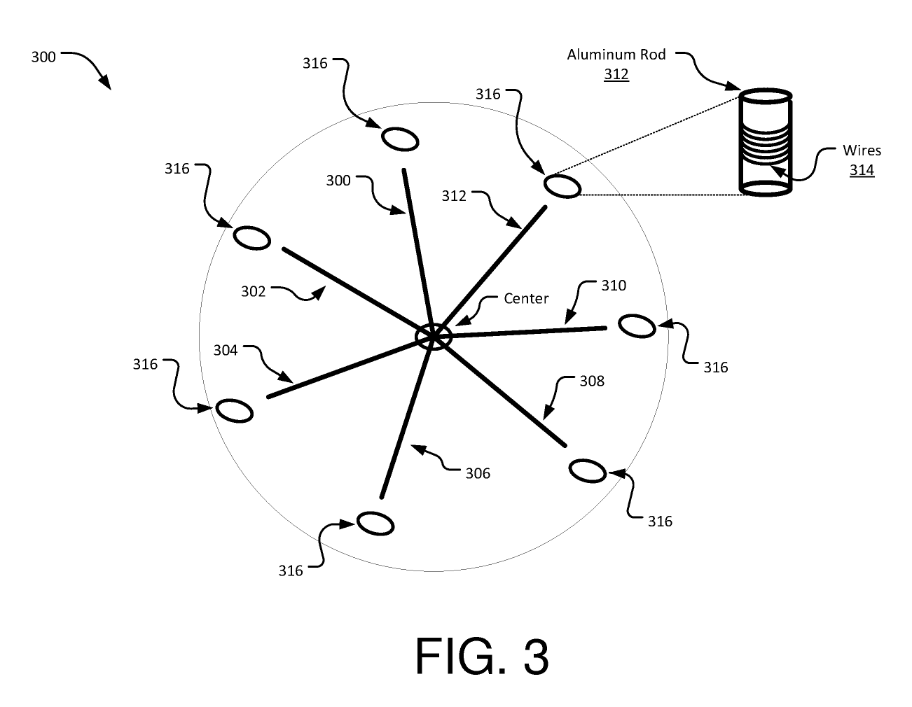

FIG. 3 shows an example generation unit 216 as described in present implementations herein. As shown, the example generation unit 216 includes seven aluminum rods 300-312 that are equidistant from a center; and a continuous loop of wrapping wires 314 and a dowel 316 for each aluminum rods 302-312. In other implementations, the rods or wires may be made of copper, silver, iron, titanium, steel, bronze, bamboo, plastic, CVP, etc. or any combination of these and any other natural or manmade products.

When combined with the crystal oscillator 212 and the magnets 214, the generation unit 216 may act as a resonating generator that manipulates frequencies that are fed into its system.

For example, each of the aluminum rods 300-312 is cut into different lengths and segments to provide a separation or manipulation of frequency waves by varying the overall lengths of the waves. Furthermore, any two adjacent aluminum rods may form the same angle (i.e., about 51 degrees in vertical, horizontal or a variation of the two states) since the aluminum rods 300-312 are equidistant to the center. In this example, the aluminum rods 300-312 have a length of 1 @ 12 inches, 2 @ 6 inches, 3 @ 4 inches, 5 @ 2.4 inches, 6 @ 2 inches, 7 @ 1.71 inches, and could increase in segments 8 @ 1.5 inches, 9 @ 1.33 inches and so on respectively. In other implementations, the rods may be smaller in length and diameter or larger depending on the application area and size.

As signal frequencies are fed into the aluminum rods 300-312, the first aluminum rod 300 may first generate a building up of energy and changes in molecular structure such as the formation of hydrogen as discussed above. From the first aluminum rod 300, smaller waves may escape to the next aluminum rod 302 and another building up of energy and changes in the molecular structure may be implemented and so on until the frequency waves reaches the seventh aluminum rod 312.

At the seventh aluminum rod 312, the frequency manipulation generates combined frequencies to form, for example a sine wave. In this example, the sine wave is clipped in order to create an imbalance (i.e., built energy is shifted from one side to the other) without breaking the continuous nature of the sine wave. The clipped sine wave generates pulses by activating sub-atomic particles (i.e., element and gases of the Periodic Charts) and thereafter radiating outward allowing molecular (elements and gases) within and surrounding the range of the interactions. The ranges vary due to density of the matter (elements and gases) in the path of the generated pulses being sent.

Going back to the process of frequency manipulation at each aluminum rod, the wrappings wires 314 in combination with the dowel 316, which is a dead tree with all fibers on it, may facilitate the generation of pulses. For example, the wrapping wires 314 creates a circuit in each of the aluminum rods 300-312 to increase frequency outputs and help create random variations in the energy signals such as energy signals fed into the booster/amplifier 210. In this example, the magnet 214, the crystal oscillator 212, and fans that produce magnetic fields of opposition in a toroidal vortex to amplify the energy signals. (not shown) may furthermore add to the imbalanced energy signals allowing the surrounding areas to seek balance by creating a point at which dialectic effects can be discharged before they reach critical mass and allow shifts in the subatomic particles. This overall setup creates a secondary electrical pulse of varying ranges that allows charging and discharging of the surrounding areas before the agglomeration of like molecules (elements and gases in the periodic charts) to force the molecules to stay in their most productive combinations.

### Example Rod Structure of Generation Unit

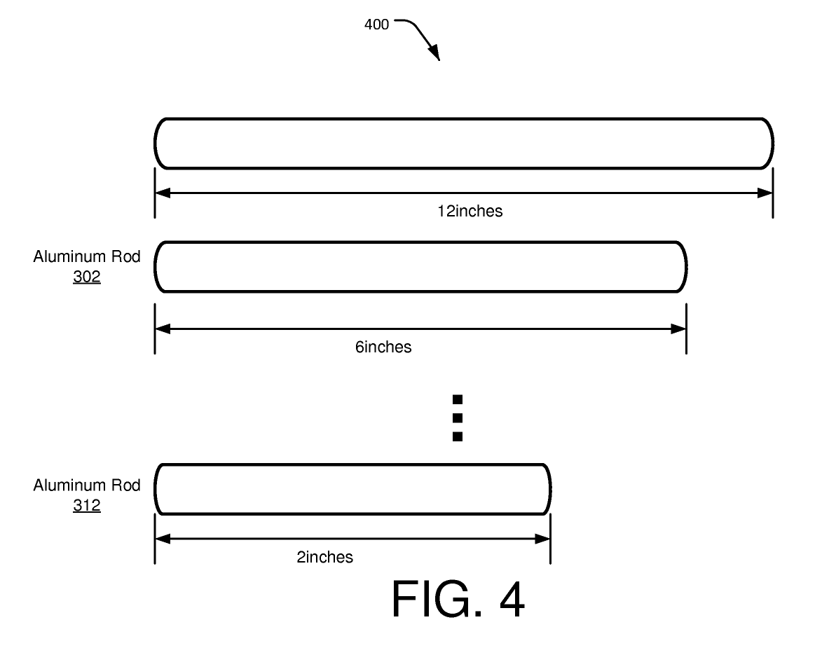

FIG. 4 shows an example rod structure 400 of the generation unit 216 as described in present implementations herein.

As shown, the aluminum rods 300-312 are configured to have different lengths to increase the frequency outputs and help create random variations in the energy signals. For example, input frequencies are first processed at the first aluminum rod 300. The processing may involve building up of the energy at the first aluminum rod 300. Smaller waves may escape to the next aluminum rod 302 and another building up of energy is implemented and so on until the frequency waves reaches the seventh aluminum rod 312 is reached. Thereafter, the generation unit 216 generates pulses at the output that may allow the device 102 to operate at a distance and not necessarily attached, for example, to the recipient 104.

For example, a harmonic energy displacement is needed to allow the device 102 to operate in the outdoors environment, over large areas and without having to use point-of-attachment to the recipient 104 being treated, whether it is human, animal, plant of inorganic matter. In this example, the configuration of the aluminum rods 300-312 may facilitate scattering of frequency that are compressed in the areas surrounding the person or matter being treated. Rather than attaching the device 102, the shifts in the energy signal being sent out into pulses may activate the surrounding in a balanced manner. That is, it forces the areas to become more centered and the energy sequence creates a balance point while releasing the imbalanced particles into the surrounding areas, which very often include imbalanced area.

### Operations for the Device

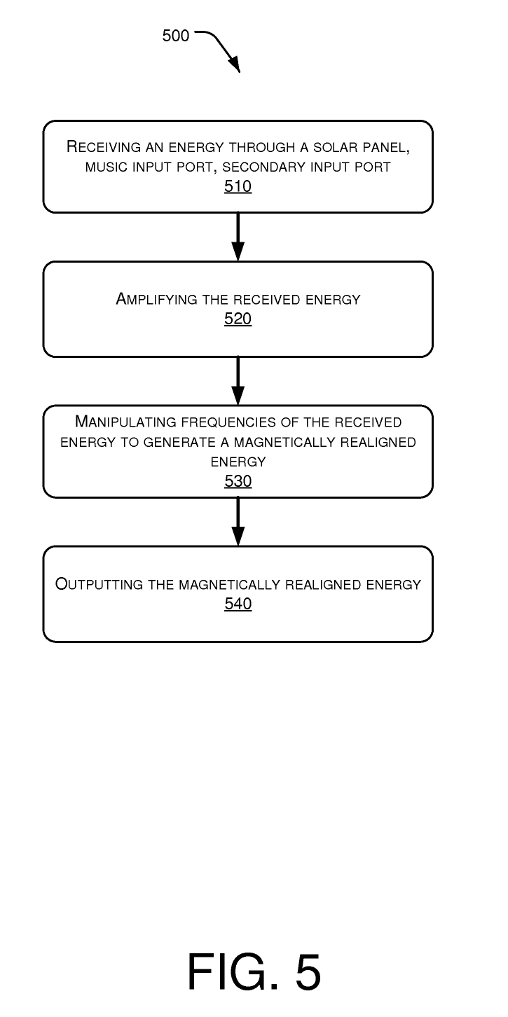

FIG. 5 illustrates an example process 500 for implementing, at least in part, the technology described herein. In particular, process 500 depicts the operations of the described herein. The process 300 is performed by, for example, the system 200.

At 510, an energy is received by the device through its solar panel, music input port, leads/iPad connector input port, and auxiliary/secondary input port.

At 520, the received energy is amplified.

At 530, the amplified received energy is exposed to a variable magnetic field to magnetically realign the received energy. For example, the generation unit 216 is made of at multiple aluminum rods of different lengths to manipulate the frequency of the amplified received energy. In either horizontal, vertical or in various angles or degrees of said angle to accomplish the best range for that application. In this example, the frequency manipulation may produce a combined frequency that is clipped to generate the magnetically realigned received energy.

At 540, outputting the magnetically realigned energy by means of a sound wave or a wired connection that is attached to an organic or inorganic material.

### Exemplary System

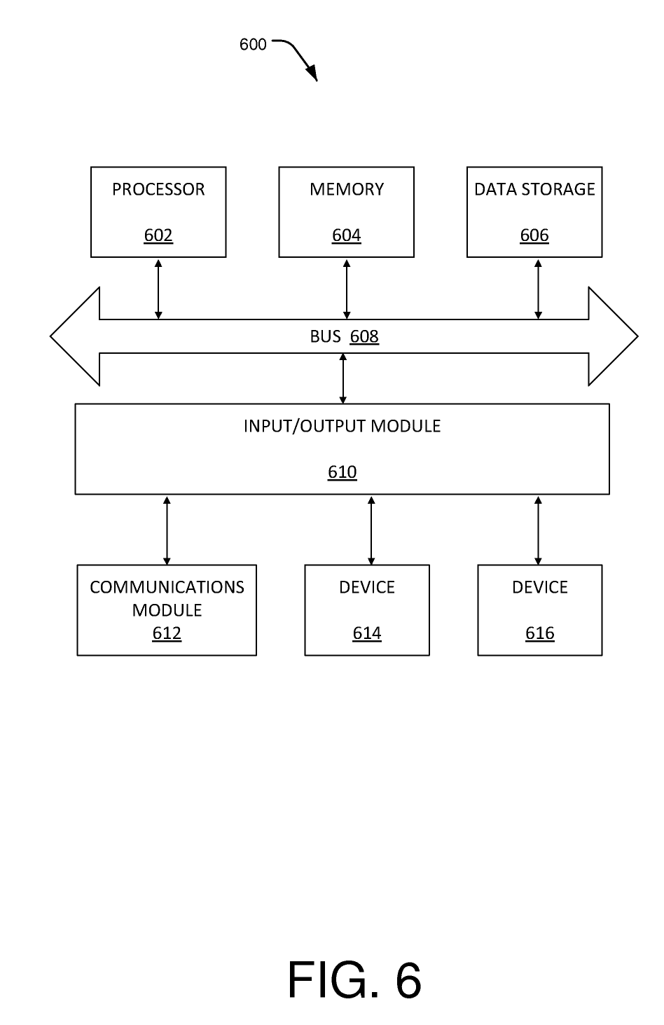

FIG. 6 is a high-level block diagram illustrating an example computer system 600 suitable for implementing the technologies described herein. In certain aspects, the computer system 600 may be implemented using hardware or a combination of software and hardware.

The illustrated computer system 600 includes a processor 602, a memory 604, and data storage 606 coupled to a bus 608 or other communication mechanism for communicating information. An input/output (I/O) module 610 is also coupled to the bus 608. A communications module 612, a device 614, and a device 616 are coupled to the I/O module 610.

The processor 602 may be a general-purpose microprocessor, a micro-controller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information. The processor 602 may be used for processing information. The processor 602 can be supplemented by, or incorporated in, special purpose logic circuitry.

The memory 604 may be Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device used for storing information, a computer program, and/or instructions to be executed by the processor 602. They memory 604 may store code that creates an execution environment for one or more computer programs used to implement technology described herein.

A computer program as discussed herein does not necessarily correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Unless indicated otherwise by the context, a module refers to a component that is hardware, firmware, and/or a combination thereof with software (e.g., a computer program.) A computer program as discussed herein does not necessarily correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

The instructions may be implemented in one or more computer program products, i.e., one or more modules of computer program instructions encoded on one or more computer readable media for execution by, or to control the operation of, the computer system 600, and according to any method well known to those of skill in the art. The term “computer-readable media” includes computer-storage media. For example, computer-storage media may include, but are not limited to, magnetic storage devices (e.g., hard disk, floppy disk, and magnetic strips), optical disks (e.g., compact disk (CD) and digital versatile disk (DVD)), smart cards, flash memory devices (e.g., thumb drive, stick, key drive, and SD cards), and volatile and non-volatile memory (e.g., random access memory (RAM), read-only memory (ROM)).

The data storage 606 may be a magnetic disk or optical disk, for example. The data storage 506 may function to store information and instructions to be used by the processor 602 and other components in the computer system 600.

The bus 608 may be any suitable mechanism that allows information to be exchanged between components coupled to the bus 508. For example, the bus 608 may be transmission media such as coaxial cables, copper wire, and fiber optics, optical signals, and the like.

The I/O module 610 can be any input/output module. Example input/output modules 610 include data ports such as Universal Serial Bus (USB) ports.

The communications module 612 may include networking interface cards, such as Ethernet cards and modems.

The device 614 may be an input device. Example devices 614 include a keyboard, a pointing device, a mouse, or a trackball, by which a user can provide input to the computer system 600.

The device 616 may be an output device. Example devices 616 include displays such as cathode ray tubes (CRT) or liquid crystal display (LCD) monitors that display information, such as web pages, for example, to the user.

### Additional and Alternative Implementation Notes

For the manipulation of frequencies as described in the device 102, other implementations of the aluminum rods 300-312 of the generation unit 216 may involve different alignment configurations. For example, the aluminum rods 300-306 are disposed in horizontal positions while the rest of the aluminum rods are disposed in vertical positions equidistant to the center as discussed above.

In the above description of exemplary implementations, for purposes of explanation, specific numbers, materials configurations, and other details are set forth in order to better explain the present invention, as claimed. However, it will be apparent to one skilled in the art that the claimed invention may be practiced using different details than the exemplary ones described herein. In other instances, well-known features are omitted or simplified to clarify the description of the exemplary implementations.

The inventor intends the described exemplary implementations to be primarily examples. The inventor does not intend these exemplary implementations to limit the scope of the appended claims. Rather, the inventor has contemplated that the claimed invention might also be embodied and implemented in other ways, in conjunction with other present or future technologies.

Moreover, the word “exemplary” is used herein to mean serving as an example, instance, or illustration. Any aspect or design described herein as exemplary is not necessarily to be construed as preferred or advantageous over other aspects or designs. Rather, use of the word “exemplary” is intended to present concepts and techniques in a concrete fashion. The term “technology,” for instance, may refer to one or more devices, apparatuses, systems, methods, articles of manufacture, and/or computer-readable instructions as indicated by the context described herein.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

These processes are illustrated as a collection of blocks in a logical flow graph, which represents a sequence of operations that can be implemented in mechanics alone or a combination with hardware, software, and/or firmware. In the context of software/firmware, the execution of the instructions on the medium may cause performance of the operations described herein. For example, or more computer-readable media with processor-executable instructions stored thereon which when executed by one or more processors may cause performance of operations described herein.

Note that the order in which the processes are described is not intended to be construed as a limitation, and any number of the described process blocks can be combined in any order to implement the processes or an alternate process. Additionally, individual blocks may be deleted from the processes without departing from the spirit and scope of the subject matter described herein.

The term “computer-readable media” is computer-storage media. For example, computer-storage media may include, but are not limited to, magnetic storage devices (e.g., hard disk, floppy disk, and magnetic strips), optical disks (e.g., compact disk [CD] and digital versatile disk [DVD]), smart cards, flash memory devices (e.g., thumb drive, stick, key drive, and SD cards), and volatile and nonvolatile memory (e.g., RAM and ROM).

What is claimed is:

1. A device comprising:
an input section configured to receive an energy;
an amplifier-booster component configured to amplify the received energy;
a generation unit configured to generate a variable magnetic field that magnetically realigns the amplified energy;
a speaker or a headphone port configured to output the magnetically realigned energy.

2. The device as recited in claim 1, wherein the input section is receiving the energy selected from a group consisting of a solar energy input, a musical input port, and a video input port.

3. The device as recited in claim 2 further comprising an equalizer component that is configured to adjust a frequency response of the received energy from the musical input port and the video input port.

4. The device as recited in claim 1, wherein the generation unit further comprises individual aluminum rods of different lengths, wherein each aluminum rod is configured to manipulate frequencies of the amplified energy.

5. The device as recited in claim 4, wherein the aluminum rod is configured to build up the received energy in a first aluminum rod, wherein smaller waves from the first aluminum rod that is received by a second aluminum rod is built up by the second aluminum rod, wherein the first and second aluminum rods are equidistant to a center of the generation unit.

6. The device as recited in claim 1, wherein the speaker or a headphone port is configured to output the magnetically realigned energy through a sound wave medium or through a wired connection that is attached to an organic or inorganic matter.

7. A method of filling a gap in a molecular structure of an organic or inorganic material, the method comprising:
receiving of an energy;
amplifying the received energy, wherein the amplified energy includes varying a frequency oscillation to generate a prism effect;
exposing the prism effect to a variable magnetic field that magnetically realigns the amplified energy;
outputting the magnetically realigned energy.

8. The method as recited in claim 7, wherein the energy is selected from a group consisting of a solar energy, an energy from a musical input, an energy from a picture input, and an energy from a video input.

9. The method as recited in claim 7, wherein the variable magnetic field is generated by manipulating frequencies of the received energy at each aluminum rod of a parallel connected generation unit.

10. The method as recited in claim 9, wherein the aluminum rod is configured to build up the received energy in a first aluminum rod, wherein smaller waves from the first aluminum rod that is received by a second aluminum rod is built up by the second aluminum rod to generate the variable magnetic field.

11. The method as recited in claim 7, wherein the outputting is implemented through a sound wave medium or through a wired connection that is attached to the organic or inorganic matter.

12. The method as recited in claim 7, wherein the magnetic realignment of the amplified energy includes an energy generation with a constant vibrational pulse rate.

13. The method as recited in claim 7 further comprising:
infusing the magnetically realigned energy to the organic or inorganic material;
determining a location of the gap based on the infused magnetically realigned energy;
in response to the determined location of the gap, charging and filling the gap with an appropriate magnetically realigned energy.

14. The method as recited in claim 13, wherein the appropriate magnetically realigned energy includes generating a sufficient amount of hydrogen atoms to fill a molecular structure area of the gap.

15. The method as recited in claim 13, wherein the gap includes a range of 0.000127-0.00000100 nanometers.

16. One or more non-transitory computer-readable media storing instructions which when executed by a machine cause the machine to perform a process comprising:
receiving of an energy;
amplifying the received energy;
manipulating frequencies of the amplified received energy, wherein the manipulating frequencies includes magnetically realigning the amplified energy;
outputting the magnetically realigned energy.

17. The method as recited in claim 16, wherein the energy is selected from a group consisting of a solar energy, an energy from a musical input, an energy from a picture input, and an energy from a video input.

18. The method as recited in claim 16, wherein the manipulating frequencies include building up of the received energy in a first aluminum rod, wherein smaller waves from the first aluminum rod that is received by a second aluminum rod is built up by the second aluminum rod, wherein the built up energies generate the magnetically realigned energy.

19. The method as recited in claim 18, wherein the magnetically realigned energy is utilized to fill up a gap in a molecular structure of an organic or inorganic matter, wherein the gap has a range of 0.000127-0.00000100 nanometers.

20. The method as recited in claim 16, wherein the outputting the magnetically realigned energy is implemented through a sound wave medium or through a wired connection that is attached to an organic or inorganic matter.
